(19) United States
(12) Patent Application Publication
(10) Pub. No.: US 2007/0264509 A1
(43) Pub. Date: Nov. 15, 2007

(54) Copolymer and Medical Device with the Copolymer

(76) Inventors: Yu-Chin Lai, Pittsford, NY (US); Weihong Lang, (US); Wenyan Yan, Fairport, NY (US)

Correspondence Address: Joseph Barrera;Bausch & Lomb Incorporated, One Bausch & Lomb Place, Rochester, NY, 14604, US

(21) Appl. No.: 11/676,599

(22) Filed: Feb. 20, 2007

Related U.S. Application Data
(63) Continuation-in-part of application No. 11/432,173, filed on May 11, 2006.

(51) Int. Cl. C08J 7/16; A61L 27/00

(52) U.S. Cl. 428/447; 428/411.1; 428/420; 428/425.5; 623/6.62

(57) ABSTRACT

A copolymer comprising monomeric units of one or more polymerizable polyalcohols selected from the groups consisting of erythritol, xylitol, sorbitol and mixtures thereof; and one or more polymerizable carboxylic acids. The invention is also directed to a medical device with a surface coating prepared with the copolymer

This application is a continuation-in-part application of U.S. patent application Ser. No. 11/432,173, filed May 11, 2006, the entire disclosure of which is incorporated herein by reference.

The invention relates to a copolymer prepared from a polymerizable polyol and a polymerizable carboxylic acid. The copolymer can be used to coat a surface of a medical device, and in particular, a surface of a contact lens.

### BACKGROUND OF THE INVENTION

The use of soft, extended-wear contact lenses have become increasingly popular over the last decade. Extended-wear contact lenses generally comprise silicone-containing polymers having high oxygen permeability (e.g., exhibiting high Dk values greater than 80) and/or high water content. These silicon-based polymers, however, tend to have hydrophobic surfaces, and consequently, have a high affinity for tear lipids and ocular proteins. The accumulation of the lipids and proteins can interfere with the clarity of the lens and cause discomfort to the patient. Hydrophilic modification of the surface can limit the accumulation of these ocular materials, and also allow provide an increase in comfort level.

One known method for modifying the hydrophobic surface of a contact lens is by treating the lens with a plasma. Plasma treatment techniques are disclosed, for example, in PCT Publications WO 96/31792 to Nicholson et al., WO 99/57581 to Chabrececk et al., and WO 94/06485 to Chatelier et al. In the Chabrececk et al. application, photoinitiator molecules are covalently bound to the surface of the article after the article has been subjected to a plasma treatment. A layer of polymerizable macromonomer is then coated onto the modified surface and heat or radiation is applied to graft polymerize the macromonomer to form the hydrophilic surface.

Other methods of permanently altering the surface properties of polymeric biomaterials, such as contact lenses include Langmuir-Blodgett deposition, controlled spin casting, chemisorptions, and vapor deposition. Examples of Langmuir-Blodgett layer systems are disclosed in U.S. Pat. Nos. 4,941,997; 4,973,429; and 5,068,318. U.S. Pat. No. 6,926,965 describes a layer-by-layer coating process fashion that involves consecutively dipping of a substrate into oppositely charged polymers until a coating of a desired thickness is formed. U.S. Pat. No. 6,630,243 describes treating a silicon-containing lens material with a plasma and then applying a hydrophilic coating to the treated surface. The coating is prepared from several different monomers including N,N-dimethylacrylamide and glycidyl methacrylate. See also, U.S. Pat. Nos. 6,599,559; 6,440,571; 6,638,563; 6,858,310 and 6,428,839.

### SUMMARY OF THE INVENTION

The invention is directed to a copolymer comprising monomeric units of one or more polymerizable polyols selected from the group consisting of polymerizable erythritol, xylitol and sorbitol; and monomeric units of one or more polymerizable carboxylic acids.

The invention is also directed to a medical device having a surface comprising a polymer coating. The polymer coating comprises monomeric units of one or more polymerizable polyols selected from the group consisting of polymerizable erythritol, xylitol and sorbitol, and monomeric units of one or more polymerizable carboxylic acids.

The invention is also directed to a medical device having a surface comprising a polymer coating. The polymer coating comprises monomeric units of one or more polymerizable polyols selected from the group consisting of polymerizable erythritol, xylitol and sorbitol, and monomeric units of one or more polymerizable carboxylic acids. Also, the medical device has a dehydration rate of less than 0.58 mg/minute and a contact angle less than 50 degrees.

### DETAILED DESCRIPTION OF THE INVENTION

The invention is directed to a copolymer comprising monomeric units of one or more polymerizable polyols selected from the group consisting of polymerizable erythritol, xylitol and sorbitol; and monomeric units of one or more polymerizable carboxylic acids. In the copolymer, the number of monomeric units of the polymerizable polyol can be greater or less than the number of monomeric units of the polymerizable carboxylic acid. The polymerizable polyols are selected from the group consisting of erythritol(meth)acrylate, xylitol(meth)acrylate, sorbitol(meth)acrylate and derivatives thereof. The term “(meth)acrylate” means methacrylate or acrylate.

The polymerizable carboxylic acids are selected from the group consisting of (meth)acrylic acid, alkenoic acids and derivatives thereof. The carboxylic acids provide the desirable negative charge associated with the polymer coating in physiologic environments. In one embodiment, the carboxylic acids are selected from alkenoic acids comprising 4 to and including 10 carbon atoms. The alkenoic acids are selected from the group consisting of maleic acid, fumaric acid, itaconic acid and derivatives thereof (such as maleic anhydride, fumaric anhydride, itaconic anhydride). The term “carboxylic acids” also includes compounds that are capable of being converted into carboxylic acids, for example, vinyldimethyloxozalone (VDMO) and corresponding anhydrides.

One of the preferred copolymers consists essentially of monomeric units of methacrylic acid or acrylic acid and monomeric units of xylitol methacrylate (such as, for example, xylitol 1-methacrylate or xylitol 3-methacrylate). Another preferred copolymer consists essentially of monomeric units of methacrylic acid or acrylic acid and monomeric units of sorbitol methacrylate. These two copolymers as well as others described in this application can be attached to the surface of the medical device by a linking compound as described herein.

The polyol(meth)acrylates (e.g., erythritol(meth)acrylate, xylitol(meth)acrylate, or sorbitol(meth)acrylate) are prepared by reacting(meth)acryloyl chloride with the desired polyol using a mole ratio of ((meth)acryloyl chloride:polyol) 1:1 or less in the presence of an acid scavenger such as triethylamine. The polyol(meth)acrylate can be separated and further purified using chromatography such as HPLC.

In one embodiment, the copolymer is attached to the surface of the medical device by a linking compound. The linking compound has functional groups that can interact with functional groups on the surface of the medical device as well as with functional groups of the copolymer. Thus, the linking compound couples the copolymer to the surface of the medical device. For example, the linking compound can comprise a functional group that can form a covalent bond with a variety of surface functional groups such as hydroxyl, mercapto, carboxyl, or amino groups. Alternatively, the linking compound can comprise an amino group that can react with carboxyl groups on the surface.

One of the preferred copolymers consists essentially of monomeric units of methacrylic acid or acrylic acid and monomeric units of xylitol methacrylate (such as, for example, xylitol 1-methacrylate or xylitol 3-methacrylate). Another preferred copolymer consists essentially of monomeric units of methacrylic acid or acrylic acid and monomeric units of sorbitol methacrylate. These two copolymers as well as others described in this application can be attached to the surface of the medical device by a linking compound, e.g., poly(DMA-co-GMA).

In one embodiment, the medical devices are contact lenses, such as extended-wear contact lenses. The modified surfaces of these contact lenses substantially prevent or limit the adsorption of tear lipids and proteins on, or the eventual absorption into, the lens, thus preserving the clarity of the contact lenses, and in turn providing a higher degree of quality and performance.

The modified surface of the medical device will preferably have an overall negative charge in a physiologic environment, and in particular, if the device is positioned on the surface of the eye as the case with a contact lens. The modified surface is best described as a polymer coating that is attached to the surface through covalent interactions.

The medical device will include a polymeric surface with functional groups on the surface. For example, silicon hydrogel polymers used to make contact lenses are generally prepared from hydrophilic monomeric units, e.g., 2-hydroxyethyl methacrylate, which provide hydroxyl surface groups, or acrylic acid or methacrylic acid monomeric units, which provide carboxyl surface groups.

If a contact lens is prepared from monomeric units of acrylic acid or methacrylic acid one preferred combination of linking compound and copolymer include poly(glycidyl methacrylate) (“poly(GMA)”) as the linking compound, and a copolymer that comprises monomeric units of methacrylic acid.

Scheme 1 depicts a general preparation route to a medical device, particularly a contact lens, with a modified surface, wherein a, b, c, a₁, a₂, c₁, and c₂ are positive integers, a=a₁+a₂, and c=c₁+c₂. The integers b and c are chosen such that the copolymer and the linking compound can be formulated in a solvent and applied to a surface of a contact lens. The integers b, b₁, and b₂ range from 10 to 10000, from 50 to 5000, from 50 to 2000, from 50 to 1000, from 10 to 1000, from 50 to 500, from 50 to 200 or from 50 to 100.

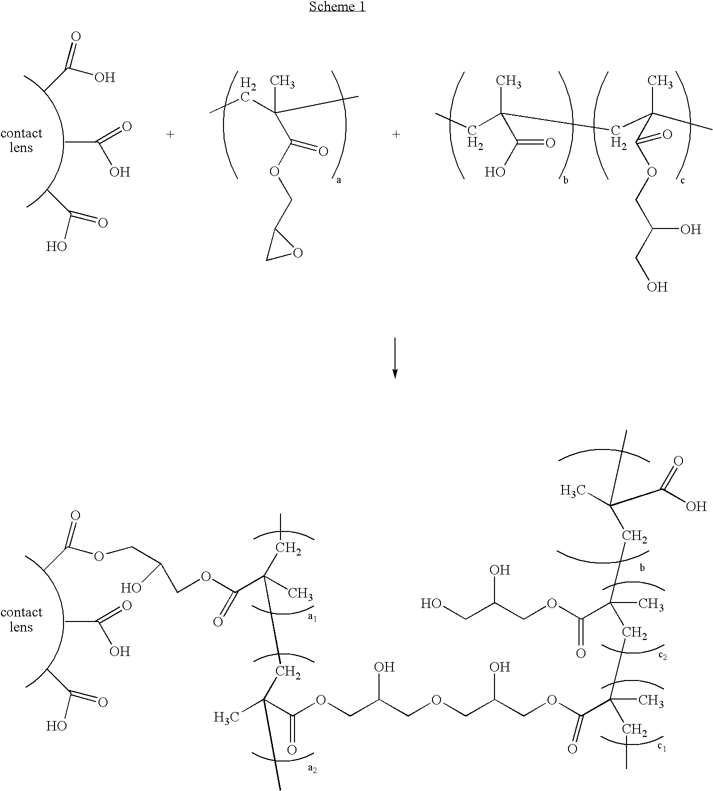

If a contact lens is prepared from monomeric units of 2-hydroxyethylmethacrylate (“HEMA”), a preferred linking compound is poly(DMA-co-GMA), and the copolymer comprises units of methacrylic acid. Scheme 2 depicts a general preparation route to a medical device, particularly a contact lens, with a modified surface, wherein u, v, x, y, b, c, c₁, and c₂ are positive integers, and c=c₁+c₂. The integers u, v, x, y, b, c, c₁, and c₂ can be chosen such that such that the copolymer and the linking compound can be formulated in a solvent and applied to a surface of a contact lens. In one embodiment, u, v, x, y, b, c, c₁, and c₂ range from 10 to 10000, from 50 to 5000, from 50 to 2000, from 50 to 1000, from 10 to 1000, from 50 to 500, from 50 to 200 or from 50 to 100.

In one embodiment, the surface treatment of the medical device is conducted at room temperature by immersing the device in a solution comprising the linking compound and the copolymer. Alternatively, a two-step coating process can be performed in which the device is first immersed in a solution containing the linking compound, and second, by then immersing the device in a solution containing the copolymer.

With regard to some polymeric materials it will be necessary to first treat the surface with a plasma discharge or corona discharge to increase the concentration of reactive functional groups at the surface. The type of gas introduced into the treatment chamber is selected to provide the desired type of reactive surface groups. For example, hydroxyl surface groups can be produced with a treatment chamber atmosphere comprising water vapor or alcohols. Carboxyl surface groups can be generated with a treatment chamber comprising oxygen or air or another oxygen-containing gas. Ammonia or amines in a treatment chamber atmosphere can generate amino surface groups. Sulfur-containing gases, such as organic mercaptans or hydrogen sulfide, can generate the mercaptan group on the surface. A combination of any of the foregoing gases also can be used in the treatment chamber. Methods and apparatuses for surface treatment by plasma discharge are disclosed in, for example, U.S. Pat. Nos. 6,550,915 and 6,794,456. The surface treated device is then contacted with the one or more coating solutions described above.

In one embodiment, the surface treatment of the medical device is conducted at room temperature by immersing the device in a solution comprising the linking compound and the copolymer. Alternatively, a two-step coating process can be performed in which the device is first immersed in a solution containing the linking compound, and second, by then immersing the device in a solution containing the copolymer.

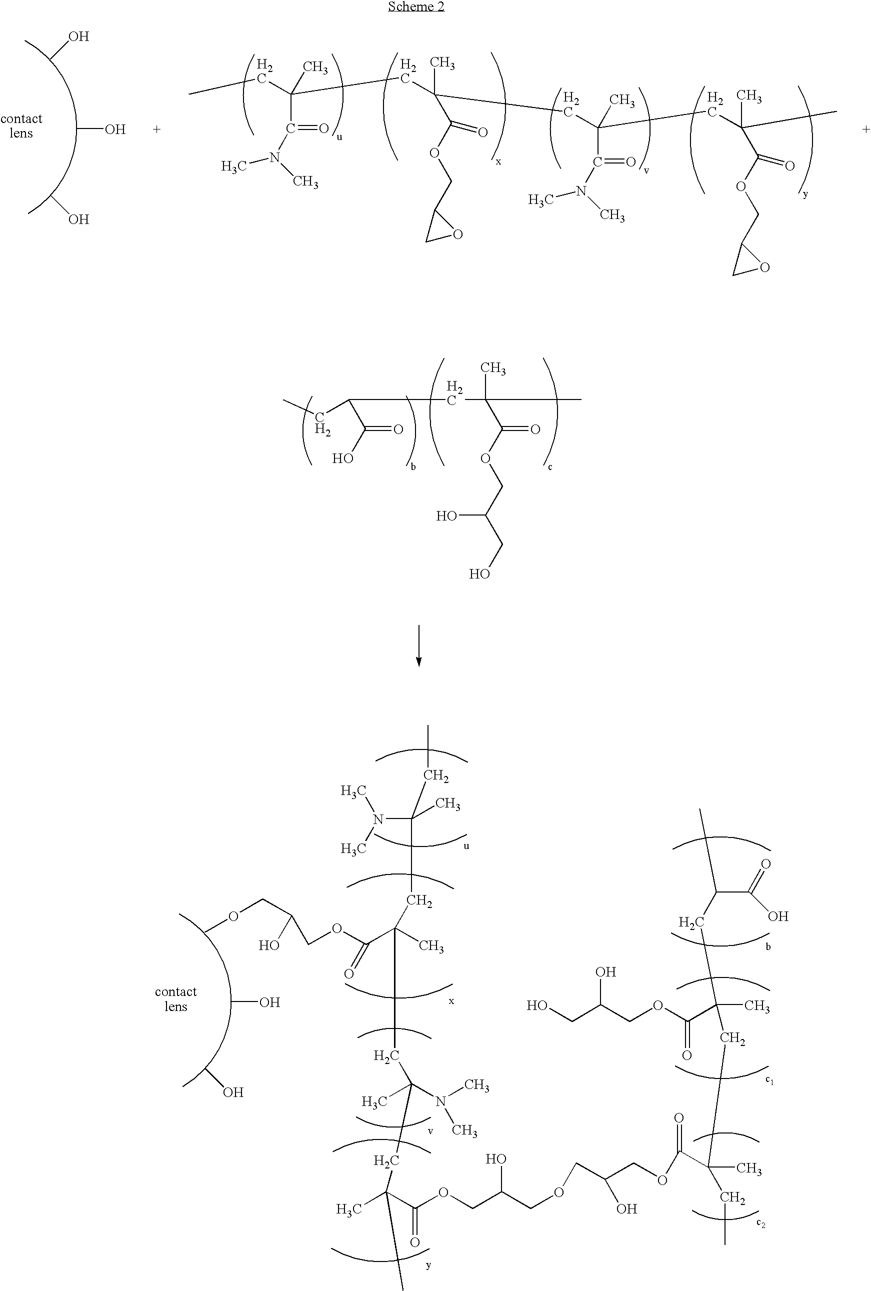

With regard to some polymeric materials it will be necessary to first treat the surface with a plasma discharge or corona discharge to increase the concentration of reactive functional groups at the surface. The type of gas introduced into the treatment chamber is selected to provide the desired type of reactive surface groups. For example, hydroxyl surface groups can be produced with a treatment chamber atmosphere comprising water vapor or alcohols. Carboxyl surface groups can be generated with a treatment chamber comprising oxygen or air or another oxygen-containing gas. Ammonia or amines in a treatment chamber atmosphere can generate amino surface groups. Sulfur-containing gases, such as organic mercaptans or hydrogen sulfide, can generate the mercaptan group on the surface. A combination of any of the foregoing gases also can be used in the treatment chamber. Methods and apparatuses for surface treatment by plasma discharge are disclosed in, for example, U.S. Pat. Nos. 6,550,915 and 6,794,456. The surface treated device is then contacted with the one or more coating solutions described above.

In one embodiment, medical device will have a dehydration rate of less than 0.6, less than a 0.59, less than 0.58, less than 0.57, less than 0.56 or less than 0.5.

In one embodiment, the polymer coating provides a contact angle of less than 90 degrees, less than 80 degrees, less than 70 degrees, less than 60 degrees or less than 50 degrees.

The invention is also directed to a method for making a medical device with a modified surface. The steps of coating a surface of a medical device are the same steps typically used to coat a polymeric surface, and thus, are well known to those of ordinary skill in the art.

In one aspect, the method comprises: (a) providing the medical device having at least a medical-device surface functional group; (b) providing a polymer having at least a hydrophilic moiety and at least a polymer functional group capable of interacting with said at least a medical-device surface functional group; and (c) contacting the medical device with the polymer at a condition sufficient to modify the surface of the medical device.

The method can further include treating the surface of the medical device to increase the concentration of the functional surface groups prior to the step of contacting the medical device with the coating composition or the coating polymer and linking compound. Again, the step of treating the surface of the medical device is conducted in a plasma discharge or corona discharge environment. A reactive gas can also be supplied to the discharge chamber to provide the desired surface functional groups.

Medical device that are in contact with body fluid, such as a wound dressing, catheters, implants (e.g., artificial hearts or other artificial organs), can be provided with the polymer coating of the invention for any number of reasons including, but not limited to, to inhibit bacterial attachment and growth or to reduce a deposit of lipids or proteins on the device.

Medical devices comprising a wide variety of polymeric materials, including hydrogel and non-hydrogel materials, can be modified to increase the hydrophilic character of the surface. In general, non-hydrogel materials are hydrophobic polymeric materials that do not contain water in their equilibrium state. Typical non-hydrogel materials comprise silicone acrylics, such as those formed from bulky silicone monomer (e.g., tris(trimethylsiloxy)silylpropyl methacrylate, commonly known as “TRIS” monomer), methacrylate end-capped poly(dimethylsiloxane) prepolymer, or silicones having fluoroalkyl side groups. On the other hand, hydrogel materials comprise hydrated, cross-linked polymeric systems containing water in an equilibrium state. Hydrogel materials contain about 5 weight percent water or more (up to, for example, about 80 weight percent). Non-limiting examples of materials suitable for the manufacture of medical devices, such as contact lenses, are herein disclosed.

Hydrogel materials for medical devices, such as contact lenses, can comprise a hydrophilic monomer, such as, HEMA, methacrylic acid (“MAA”), acrylic acid (“AA”), methacrylamide, acrylamide, N,N′-dimethylmethacrylamide, or N,N′-dimethylacrylamide; copolymers thereof; hydrophilic prepolymers, such as poly(alkylene oxide) having varying chain length, functionalized with polymerizable groups; and/or silicone hydrogels comprising siloxane-containing monomeric units and at least one of the aforementioned hydrophilic monomers and/or prepolymers. Hydrogel materials also can comprise a cyclic lactam, such as N-vinyl-2-pyrrolidone (“NVP”), or derivatives thereof. Still further examples are the hydrophilic vinyl carbonate or vinyl carbamate monomers disclosed in U.S. Pat. No. 5,070,215, and the hydrophilic oxazolone monomers disclosed in U.S. Pat. No. 4,910,277. Other suitable hydrophilic monomers will be apparent to one skilled in the art.

Silicone hydrogels generally have water content greater than about 5 weight percent and more commonly between about 10 to about 80 weight percent. Such materials are usually prepared by polymerizing a mixture containing at least one siloxane-containing monomer and at least one hydrophilic monomer. Typically, either the siloxane-containing monomer or the hydrophilic monomer functions as a crosslinking agent (a crosslinking agent or crosslinker being defined as a monomer having multiple polymerizable functionalities) or a separate crosslinker may be employed. Applicable siloxane-containing monomeric units for use in the formation of silicone hydrogels are known in the art and numerous examples are provided, for example, in U.S. Pat. Nos. 4,136,250; 4,153,641; 4,740,533; 5,034,461; 5,070,215; 5,260,000; 5,310,779; and 5,358,995.

Examples of applicable siloxane-containing monomeric units include bulky polysiloxanylalkyl(meth)acrylic monomers. The term “(meth)acrylic” means methacrylic or acrylic, depending on whether the term “meth” is present or absent. An example of bulky polysiloxanylalkyl(meth)acrylic monomers are represented by the following Formula I:

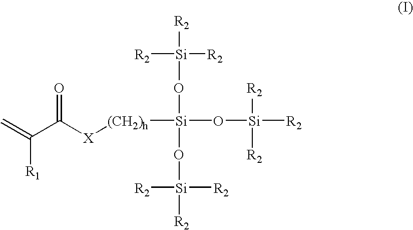

wherein X denotes —O— or —NR—; each R₁ independently denotes hydrogen or methyl; each R₂ independently denotes a lower alkyl radical, phenyl radical or a group represented by

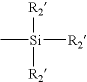

wherein each R′₂ independently denotes a lower alkyl, fluoroalkyl, or phenyl radical; and h is 1 to 10. The term “lower alkyl” means an alkyl radical having 1, 2, 3, 4, 5, 6, 7, 8, 9, or 10 carbon atoms, such as methyl, ethyl, propyl, butyl, isobutyl, pentyl, isopentyl, or hexyl radical.

A suitable bulky monomer is methacryloxypropyltris(trimethyl-siloxy)silane or tris(trimethylsiloxy)silylpropyl methacrylate (“TRIS”).

Another class of representative silicon-containing monomers includes silicone-containing vinyl carbonate or vinyl carbamate monomers such as: 1,3-bis{4-vinyloxycarbonyloxy)but-1-yl}tetramethyldisiloxane; 3-(trimethylsilyl)propyl vinyl carbonate; 3-(vinyloxycarbonylthio)propyl-{tris(trimethylsiloxy)silane}; 3-{tris(trimethylsiloxy)silyl}propyl vinyl carbamate; 3-{tris(trimethylsiloxy)silyl}propyl allyl carbamate; 3-{tris(trimethylsiloxy)silyl}propyl vinyl carbonate; t-butyldimethylsiloxyethyl vinyl carbonate; trimethylsilylethyl vinyl carbonate; and trimethylsilylmethyl vinyl carbonate.

Another class of representative silicon-containing monomers includes silicone-containing vinyl carbonate or vinyl carbamate monomers such as: 1,3-bis{4-vinyloxycarbonyloxy)but-1-yl}tetramethyl-disiloxane; 3-(trimethylsilyl)propyl vinyl carbonate; 3-(vinyloxycarbonylthio)propyl-{tris(trimethylsiloxy)silane}; 3-{tris(trimethylsiloxy)silyl}propyl vinyl carbamate; 3-{tris(trimethylsiloxy)silyl}propyl allyl carbamate; 3-{tris(trimethylsiloxy)silyl}propyl vinyl carbonate; t-butyldimethylsiloxyethyl vinyl carbonate; trimethylsilylethyl vinyl carbonate; and trimethylsilylmethyl vinyl carbonate.

An example of silicon-containing vinyl carbonate or vinyl carbamate monomers are represented by Formula II:

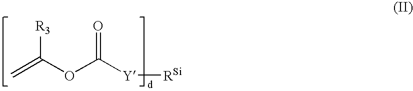

wherein:

Y′ denotes —O—, —S— or —NH—;

R^{Si} denotes a silicon-containing organic radical;

R₃ denotes hydrogen or methyl; and

d is 1, 2, 3 or 4.

Suitable silicon-containing organic radicals R^{Si} include the following:

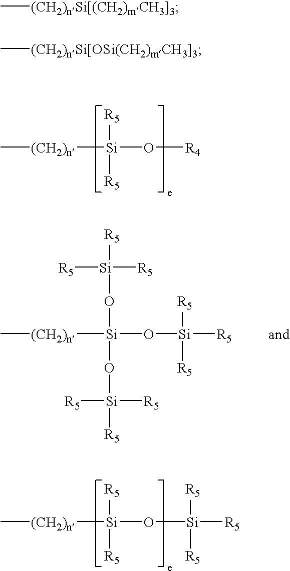

wherein

R₄ denotes

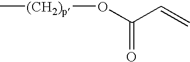

wherein p′ is from 1 to and including 6;

R₅ denotes an alkyl radical or a fluoroalkyl radical having from 1 to and including 6 carbon atoms;

e is 1 to 200; n′ is 1, 2, 3 or 4; and m′ is 0, 1, 2, 3, 4 or 5.

An example of a particular species within Formula II is represented by Formula III.

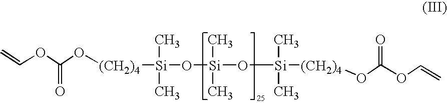

Another class of silicon-containing monomer includes polyurethane-polysiloxane macromonomers (also sometimes referred to as prepolymers), which may have hard-soft-hard blocks like traditional urethane elastomers. They may be end-capped with a hydrophilic monomer such as HEMA. Examples of such silicone urethanes are disclosed in a variety or publications, including Lai, Yu-Chin, “The Role of Bulky Polysiloxanylalkyl Methacryates in Polyurethane-Polysiloxane Hydrogels,” Journal of Applied Polymer Science, Vol. 60, 1193-1199 (1996). PCT Published Application No. WO 96/31792 discloses examples of such monomers, which disclosure is hereby incorporated by reference in its entirety. Further examples of silicone urethane monomers are represented by Formulae IV and V:

E(*D*A*D*G)ₐ*D*A*D*E′ (IV)

or

E(*D*G*D*A)ₐ*D*G*D*E′ (V),

wherein:

D denotes an alkyl diradical, an alkyl cycloalkyl diradical, a cycloalkyl diradical, an aryl diradical or an alkylaryl diradical having 6 to 30 carbon atoms;

G denotes an alkyl diradical, a cycloalkyl diradical, an alkyl cycloalkyl diradical, an aryl diradical or an alkylaryl diradical having 1 to 40 carbon atoms and which may contain ether, thio or amine linkages in the main chain;

* denotes a urethane or ureylene linkage;

a is at least 1;

A denotes a divalent polymeric radical of Formula VI:

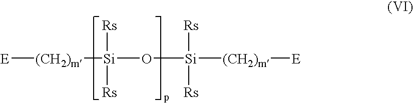

wherein:
each Rₛ independently denotes an alkyl or fluoro-substituted alkyl group having 1 to 10 carbon atoms which may contain ether linkages between carbon atoms;
m′ is at least 1; and
p is a number which provides a moiety weight of 400 to 10,000;
each of E and E′ independently denotes a polymerizable unsaturated organic radical represented by Formula VII:

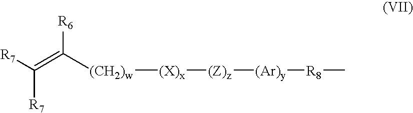

wherein:

R₆ is hydrogen or methyl;

R₇ is hydrogen, an alkyl radical having from 1 to and including 6 carbon atoms, or a —CO—Y—R₉ radical wherein Y is —O—, —S— or —NH—;

R₈ is a divalent alkylene radical having from 1 to and including 10 carbon atoms;

R₉ is a alkyl radical having from 1 to and including 12 carbon atoms;

X denotes —CO— or —OCO—;

Z denotes —O— or —NH—;

Ar denotes a substituted or unsubstituted aromatic radical having from 6 to and including 30 carbon atoms;

w is from 0 to and including 6; x is 0 or 1; y is 0 or 1; and z is 0 or 1.

A more specific example of a silicone-containing urethane monomer is represented by Formula VIII:

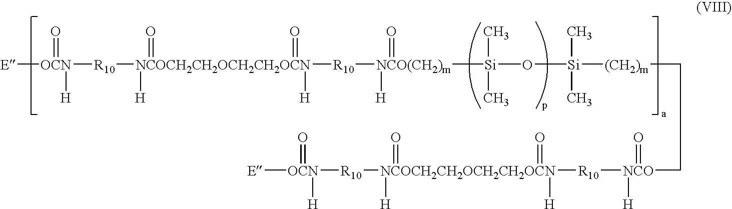

wherein m is at least 1 and is preferably 3 or 4, a is at least 1 and preferably is 1, p is a number which provides a moiety weight of 400 to 10,000 and is preferably at least 30, R₁₀ is a diradical of a diisocyanate after removal of the isocyanate group, such as the diradical of isophorone diisocyanate, and each E″ is a group represented by:

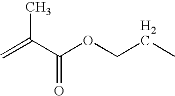

A preferred silicone hydrogel material comprises (in the bulk monomer mixture that is copolymerized) 5 to 50 percent, preferably 10 to 25, by weight of one or more silicone macromonomers, 5 to 75 percent, preferably 30 to 60 percent, by weight of one or more poly(siloxanylalkyl(meth)acrylic)monomers, and 10 to 50 percent, preferably 20 to 40 percent, by weight of a hydrophilic monomer. In general, the silicone macromonomer is a poly(organosiloxane) capped with an unsaturated group at two or more ends of the molecule. In addition to the end groups in the above structural formulas, U.S. Pat. No. 4,153,641 to Deichert et al. discloses additional unsaturated groups, including acryloxy or methacryloxy. Fumarate-containing materials such as those taught in U.S. Pat. Nos. 5,512,205; 5,449,729; and 5,310,779 to Lai are also useful substrates in accordance with the invention. Preferably, the silane macromonomer is a silicon-containing vinyl carbonate or vinyl carbamate or a polyurethane-polysiloxane having one or more hard-soft-hard blocks and end-capped with a hydrophilic monomer.

In particular regard to contact lenses, the fluorination of certain monomers used in the formation of silicone hydrogels has been indicated to reduce the accumulation of deposits on contact lenses made therefrom, as described in U.S. Pat. Nos. 4,954,587, 5,079,319 and 5,010,141. Moreover, the use of silicone-containing monomers having certain fluorinated side groups (e.g., —(CF₂)—H) have been found to improve compatibility between the hydrophilic and silicone-containing monomeric units, as described in U.S. Pat. Nos. 5,387,662 and 5,321,108.

In another aspect, a polymeric material of the present invention comprises an additional monomer selected from the group consisting of hydrophilic monomers and hydrophobic monomers.

Hydrophilic monomers can be nonionic monomers, such as 2-hydroxyethyl methacrylate (“HEMA”), 2-hydroxyethyl acrylate (“HEA”), 2-(2-ethoxyethoxy)ethyl(meth)acrylate, glyceryl(meth)acrylate, poly(ethylene glycol(meth)acrylate), tetrahydrofurfuryl(meth)acrylate, (meth)acrylamide, N,N′-dimethylmethacrylamide, N,N′-dimethylacrylamide (“DMA”), N-vinyl-2-pyrrolidone (or other N-vinyl lactams), N-vinyl acetamide, and combinations thereof. Other hydrophilic monomers can have more than one polymerizable group, such as tetraethylene glycol(meth)acrylate, triethylene glycol(meth)acrylate, tripropylene glycol(meth)acrylate, ethoxylated bisphenol-A (meth)acrylate, pentaerythritol(meth)acrylate, pentaerythritol(meth)acrylate, ditrimethylolpropane(meth)acrylate, ethoxylated trimethylolpropane(meth)acrylate, dipentaerythritol(meth)acrylate, alkoxylated glyceryl(meth)acrylate. Still further examples of hydrophilic monomers are the vinyl carbonate and vinyl carbamate monomers disclosed in U.S. Pat. No. 5,070,215, and the hydrophilic oxazolone monomers disclosed in U.S. Pat. No. 4,910,277. The contents of these patents are incorporated herein by reference. The hydrophilic monomer also can be an anionic monomer, such as 2-methacryloyloxyethylsulfonate salts. Substituted anionic hydrophilic monomers, such as from acrylic and methacrylic acid, can also be utilized wherein the substituted group can be removed by a facile chemical process. Non-limiting examples of such substituted anionic hydrophilic monomers include trimethylsilyl esters of (meth)acrylic acid, which are hydrolyzed to regenerate an anionic carboxyl group. The hydrophilic monomer also can be a cationic monomer selected from the group consisting of 3-methacrylamidopropyl-N,N,N-trimethyammonium salts, 2-methacryloyloxyethyl-N,N,N-trimethylammonium salts, and amine-containing monomers, such as 3-methacrylamidopropyl-N,N-dimethyl amine. Other suitable hydrophilic monomers will be apparent to one skilled in the art.

Non-limiting examples of hydrophobic monomers are C₁-C₂₀ alkyl and C₃-C₂₀ cycloalkyl(meth)acrylates, substituted and unsubstituted aryl(meth)acrylates (wherein the aryl group comprises 6 to 36 carbon atoms), (meth)acrylonitrile, styrene, lower alkyl styrene, lower alkyl vinyl ethers, and C₂-C₁₀ perfluoroalkyl(meth)acrylates and correspondingly partially fluorinated (meth)acrylates.

Solvents useful in the surface treatment of the medical device, such as a contact lens, include solvents that readily solubilize the polymers such as water, alcohols, lactams, amides, cyclic ethers, linear ethers, carboxylic acids, and combinations thereof. Preferred solvents include tetrahydrofuran (“THF”), acetonitrile, N,N-dimethyl formamide (“DMF”), and water. The most preferred solvent is water.

### EXAMPLE 1

### Preparation of Copolymer of Glyceryl Methacrylate and Acrylic Acid

A 250-ml three-neck flask was equipped with a stirrer and a condenser. The flask was immersed in an oil bath. Into this flask were added 100 ml of deionized water, 6.207 g (or 38.75 mmol) of glyceryl methacrylate, 1.385 g (or 19.22 mmol) of acrylic acid and 0.090 g (or 0.55 mmol) of AlBN polymerization initiator. The contents of the flask were bubbled with nitrogen vigorously for 20 minutes while under stirring, then the nitrogen flow was turned to a lower rate. The contents of the flask were heated to and kept at 70° C. under nitrogen purging for two days. The copolymer was saved as a 3% (by weight) solution in deionized water.

### EXAMPLE 2

### Lens Casting of Polyurethane-Siloxane Hydrogel Formulation

Monomer mix consisting of I4D6S5H (further described below)/TRIS/NVP(N-vinyl pyrrolidone)/DMA/HEMAVC (HEMA vinyl carbamate)/n-hexanol/Darocur™ 1173 (a photopolymerization initiator)/IMVT (1,4-bis(2-metharylamido)anthraquinone, providing a blue tint) at weight ratio of 60/15/22/7/2.5/1/10/0.5/150 ppm was made. I4D6S5H is a prepolymer having a formula of HEMA-IPDI-(PDMS5000-IPDI-DEG-IPDI)₄(PDMS5000-IPDI)₂-HEMA, wherein IPDI is isophoronediisocyanate, PDMS5000 is polydimethysiloxane having a molecular weight of 5000, and DEG is diethylene glycol. The mix was used to cast lenses using polypropylene molds. Lenses were released from the molds, extracted with isopropanol overnight, and then placed in deionized water.

### EXAMPLE 3

### Surface Treatment with Copolymer of Glyceryl Methacrylate and Acrylic Acid

An aqueous solution containing 3 weight percent of the copolymer produce in Example 1 was prepared. Lenses from Example 2 were placed in vials containing this solution and then autoclaved for one thirty-minute cycle. Five lenses from Example 2 were also autoclave in deionized water. After autoclaving, the lenses were inspected for wettability. Lenses treated with the copolymer solution were found to be more wettable and lubricious than those that were untreated or treated only with deionized water. When the lenses treated with the copolymer solution were placed in borate buffered saline, they became qualitatively even more wettable and lubricious, as judged by rubbing.

### EXAMPLE 4

### Dehydration Test

Both untreated and treated lenses were subjected to a dehydration test. Untreated and treated lenses were desalinated, then placed in deionized water before being evaluated for dehydration. Dehydration tests were carried out using a TA Instruments Q50 thermal gravimetric analyzer (“TGA”). A disc of approximately 7 mm in diameter was punched from the center of a lens. The disc was dabbed with Kimwipes® to remove any surface water and then placed on the TGA balance. The balance was enclosed in a chamber under a dry nitrogen pure (at 60 ml/minute flow rate). The sample was ramped at 50° C./minute up to 37° C. and was held isothermally. Mass loss versus time was monitored and the test was terminated when the mass loss rate (in %/minute) was less than 0.05. The dehydration rate was calculated as rate of mass loss between 60 and 20 percent per minute and reported as mg/minute. The results of the wettability and dehydration test are shown in Table 1 as the average of five measurements.

TABLE 1

| | Lens | | | | |
|---|---|---|---|---|---|
| | A | B | C | D | E |
| Water Content | 37.8 | 39.3 | 43.9 | 41.4 | 34.8 |
| (weight %) | | | | | |
| Contact Angle | 118 | 104 | 45 | 41 | 90 |
| (degrees) | | | | | |
| Time to 95% | 8.2 | 7.4 | 9.6 | 6.1 | 4.3 |
| Weight Loss | | | | | |
| Dehydration Rate | 0.601 | 0.624 | 0.557 | 0.658 | 0.644 |
| (mg/min.) | | | | | |

Notes:
A = control lens as described in Example 2, untreated
B = treated with copolymer of glyceryl methacrylate and acrylic acid (4:1 mole ratio)
C = treated with copolymer of glyceryl methacrylate and acrylic acid (2:1 mole ratio, pH = 3–4)
D = treated with solution containing only poly(acrylic acid)
E = commercial PureVison ® lens (Bausch & Lomb Incorporated, Rochester, New York)

### EXAMPLE 5

### Coating on Another Type of Lenses

Lenses manufactured with the same formulation as commercial PureVision® lenses (Bausch & Lomb Incorporated, Rochester, N.Y.) were treated with air plasma, extracted, hydrated, and autoclaved in a solution containing 0.3% (by weight) poly(glycerol methacrylate-co-acrylic acid) (2:1 molar ratio) and 1% (by weight) poly(DMA-co-GMA) (68:32 molar ratio) in MOPS (3-{N-morpholino}propanesulfonic acid) buffer. After autoclaving, the lenses were rinsed with deionized water and saved in borate buffer saline (“BBS”). The lenses were again soaked in and rinsed with deionized water immediately before the dehydration test (same procedure as in Example 4). Table 2 shows a comparison between commercial PureVision® lenses and experimental lenses of this Example.

TABLE 2

| | | | | | | Average |
|---|---|---|---|---|---|---|
| | | | | | Dehydration | Water |
| | XPS Analysis | | | | Rate | Content |
| Lenses | C₁ₛ | N₁ₛ | O₁ₛ | Si₂ₚ | (mg/min.) | (% wt.) |
| Commercial | 65.3 (0.4) | 8.0 (0.2) | 19.4 (0.2) | 7.0 (0.2) | 0.644 | 34.8 |
| PureVision ® | | | | | | |
| This | 66.1 (0.5) | 6.2 (0.4) | 25.4 (0.5) | 1.3 (0.4) | 0.558 | 39.9 |
| Example | | | | | | |

The results show that the coated lenses were covered well with the coating polymer, as evidenced by the significant reduction in surface silicon and nitrogen. The coated lenses show significant reduction in water dehydrate rate.

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A copolymer comprising a monomeric unit of one or more polymerizable polyols selected from the group consisting of polymerizable erythritol, xylitol and sorbitol; and a monomeric unit of one or more polymerizable carboxylic acids.

2. The copolymer of claim 1, wherein the polymerizable polyols are selected from the group consisting of erythritol(meth)acrylate, xylitol(meth)acrylate, sorbitol(meth)acrylate and derivatives thereof.

3. The copolymer of claim 1, wherein the polymerizable carboxylic acids are selected from the group consisting of (meth)acrylic acid, alkenoic acids and derivatives thereof.

4. A medical device having a surface comprising a polymer coating, wherein the polymer coating comprises monomeric units of one or more polymerizable polyols selected from the group consisting of polymerizable erythritol, xylitol and sorbitol; and monomeric units of one or more polymerizable carboxylic acids.

5. The medical device of claim 4, wherein the polymerizable carboxylic acids are selected from the group consisting of (meth)acrylic acid, alkenoic acids and derivatives thereof.

6. The medical device of claim 4, wherein the polymerizable polyols are selected from the group consisting of erythritol(meth)acrylate, xylitol(meth)acrylate, sorbitol(meth)acrylate and derivatives thereof.

7. The medical device of claim 4, wherein the polymer coating is attached to the surface of the medical device through a linking compound, wherein the linking compound comprises; (a) a first functional group that interacts with a surface functional group of the medical device, and (b) a second functional group that interacts with a functional group of the polymer coating.

8. The medical device of claim 4, wherein the medical device has a dehydration rate of less than about 0.6 mg/minute.

9. The medical device of claim 4, wherein the medical device has a dehydration rate of less than about 0.58 mg/minute.

10. The medical device of claim 4, wherein the medical device has a contact angle of less than about 90 degrees.

11. The medical device of claim 8, wherein the medical device has a contact angle of less than about 70 degrees.

12. The medical device of claim 9, wherein the medical device has a contact angle of less than about 50 degrees.

13. The medical device of claim 7, wherein the medical device has a contact angle of less than about 50 degrees.

14. The medical device of claim 4, wherein the coating polymer provides an overall negative charge in a physiological environment.

15. The medical device of claim 4, wherein the polymer coating further comprises one or more hydrophilic monomeric units selected from the group consisting of neutral monomers, cationic monomers and ampholytic monomers.

16. The medical device of claim 15, wherein the neutral monomers are selected from the group consisting of N,N-dimethylacrylamide, N-vinylpyrrolidone, (meth)acrylamide, 2-hydroxyethyl methacrylate and glyceryl methacrylate.

17. The medical device of claim 4, wherein the medical device comprises a polysiloxane.

18. The medical device of claim 17, wherein the medical device is a contact lens.

19. A medical device with a surface comprising a polymer coating that is attached to the surface, the polymer coating comprising monomeric units of one or more polymerizable polyols selected from the group consisting of erythritol(meth)acrylate, xylitol(meth)acrylate, sorbitol(meth)acrylate and derivatives thereof, and monomeric units of one or more polymerizable carboxylic acids, wherein the medical device has a dehydration rate of less than 0.58 mg/minute and a contact angle less than 50 degrees.

20. The medical device of claim 19, wherein the polymer coating is covalently attached to surface functional groups that are formed by plasma discharge or a corona discharge.

21. The medical device of claim 19, wherein the polymer coating is attached to the surface of the medical device through a linking compound, wherein the linking compound comprises; (a) a first functional group that interacts with a surface functional group of the medical device, and (b) a second functional group that interacts with a functional group of the polymer coating.

22. A method for making a contact lens, the method comprising:
providing a surface of a contact lens with a plurality of surface functional groups;
contacting the surface with a coating composition, the coating composition comprising a copolymer with monomeric units of a polymerizable polyalcohol selected from one or more group consisting of polymerizable erythritol, xylitol, sorbitol and mixtures thereof; and monomeric units of a polymerizable carboxylic acid.

23. The method of claim 22, further comprising subjecting the surface of the contact lens in a plasma discharge or corona discharge to increase concentration of surface functional groups prior to the step of contacting the surface with the coating composition.
